(19) United States
(12) Patent Application Publication
(10) Pub. No.: US 2006/0067970 A1
(43) Pub. Date: Mar. 30, 2006

(54) Method for producing decalcified hard tissue sample

(75) Inventors: Hiroyuki Okihana, Tokyo (JP); Masahiro Mukaida, Saitama (JP)

Correspondence Address: GREENBLUM & BERNSTEIN, P.L.C., 1950 ROLAND CLARKE PLACE, RESTON, VA, 20191, US

(73) Assignee: PENTAX Corporation, Tokyo (JP)

(21) Appl. No.: 11/236,495

(22) Filed: Sep. 28, 2005

(30) Foreign Application Priority Data
Sep. 28, 2004 (JP) 2004-281642

(51) Int. Cl. A61F 2/00

(52) U.S. Cl. 424/423

(57) ABSTRACT

A method for producing a decalcified hard tissue sample by embedding a hard tissue in a liquid-penetration-permitting resin, and then decalcifying it.

### FIELD OF THE INVENTION

The present invention relates to a method for producing a decalcified hard tissue sample, particularly to a method for producing a decalcified sample of a living hard tissue replacement, a living hard tissue or their composite, to which cells are attached, simply at a low cost.

### BACKGROUND OF THE INVENTION

To microscopically observe hard tissues such as composites comprising scaffolds constituted by living hard tissue replacements, to which cells are attached, living hard tissues, etc., samples of 10 μm or less in thickness are usually produced. Such samples have conventionally been produced by decalcifying hard tissues, embedding them in paraffin, slicing them, and staining them. Particularly when living body components are contained in small amounts, however, this method has difficulty in observing the true conditions of cells and the overall hard tissue structures, because the hard tissues are too weak or lost after the calcium components are dissolved away. In an artificial bone, to which cells are attached and cultured, for instance, it does not have sufficient scaffold because of too little collagen, etc. after decalcification, failing to sufficiently keep the conditions of the attached cells.

Investigation has thus been conducted to provide a method of embedding a hard tissue in a resin without decalcification, and slicing and staining it. This method, however, can produce only as thick sections as about 100 μm, needing the grinding of the sections, and thus resulting in a high sample production cost. In addition, it suffers the problem that the number of samples obtained from a specimen of the same size is 1/100 or less that of decalcified samples.

As a method for producing a sliced sample of a non-decalcified hard tissue, JP 2000-346770 A proposes a method of embedding a non-decalcified hard tissue in an ethylenically unsaturated monomer (for instance, MMA), and an azo-type polymerization initiator capable of polymerizing the monomer at low temperatures, polymerizing the ethylenically unsaturated monomer, and slicing the hard tissue. Because the ethylenically unsaturated monomer has excellent permeability to the hard tissue, specimens embedded in such monomer have excellent sliceability. However, because an embedded hard tissue is sliced in the method of JP 2000-346770 A, the fine structure of the hard tissue would likely be broken by slicing if the hard tissue had high hardness. In addition, embedding takes 3-4 weeks in this method.

As a method for producing a sliced sample while keeping the conditions of a tissue well, JP 2002-31586 A proposes a method of immersing the hard tissue in an aqueous solution of carboxymethylcellulose, etc., freezing it, placing the resultant frozen, embedded specimen on a supporting block via glycerin, attaching a thin plastic film coated with an adhesive to a surface of the specimen, and horizontally slicing the frozen, embedded specimen. Though the method of JP 2002-31586 A works well on a soft tissue sample, it destroys the fine structure of a hard tissue during slicing. In addition, it needs a freeze-slicing apparatus, resulting in a high cost.

### OBJECT OF THE INVENTION

Accordingly, an object of the present invention is to provide a method for producing a decalcified hard tissue sample simply at a low cost while keeping the fine structure of the hard tissue.

### DISCLOSURE OF THE INVENTION

As a result of intense research in view of the above object, the inventors have found that decalcification after embedding a hard tissue in a liquid-penetration-permitting resin can produce a decalcified hard tissue sample simply at a low cost while keeping the fine structure of the hard tissue. The present invention has been completed based on this finding.

Thus, the method for producing a decalcified hard tissue sample according to the present invention comprises embedding the hard tissue in a liquid-penetration-permitting resin, and then decalcifying it. The decalcified, embedded hard tissue is preferably re-embedded in a resin.

In a preferred embodiment of the present invention, the hard tissue is preferably a living hard tissue, a first composite comprising a scaffold constituted by a living hard tissue replacement and cells, a second composite comprising the scaffold and the living hard tissue, or a third composite comprising the scaffold, the cells and the living hard tissue. The living hard tissue replacement is preferably a calcium compound, more preferably hydroxyapatite. The cells are preferably motor cells, which are more preferably at least one selected from the group consisting of osteoblasts, osteoblast-like cells, bone cells, cartilage cells, muscle cells, and their stem cells, precursor cells and tumor cells.

The monomer of the liquid-penetration-permitting resin is preferably hydroxyalkyl(meth)acrylate, more preferably 2-hydroxyethyl methacrylate.

### BRIEF DESCRIPTION OF THE DRAWINGS

FIG. 1 is a cross-sectional view showing a dehydrated hard tissue piece, which is immersed in a primary immersion liquid.

FIG. 2 is a cross-sectional view showing a primarily immersed hard tissue piece, which is immersed in a secondary immersion liquid.

FIG. 3 is a cross-sectional view showing a secondarily immersed hard tissue piece, which is embedded in a liquid-penetration-permitting resin.

FIG. 4 is a cross-sectional view showing a specimen having an embedded hard tissue piece.

FIG. 5 is a cross-sectional view showing an embedded specimen, which is immersed in a decalcifying liquid.

FIG. 6 is a cross-sectional view showing a decalcified specimen.

FIG. 7 is a cross-sectional view showing a decalcified specimen, which is re-embedded in a resin.

FIG. 8 is a cross-sectional view showing a re-embedded specimen.

FIG. 9 is a cross-sectional view showing a re-embedded specimen, which is placed in a mold and adhered to a supporting block.

FIG. 10 is a cross-sectional view showing the re-embedded specimen fixed to the supporting block.

FIG. 11 is a photomicrograph (magnification: 4 times) showing the cell-containing, decalcified hydroxyapatite sample of Example 1.

FIG. 12 is a photomicrograph (magnification: 20 times) showing a portion of the sample of Example 1.

FIG. 13 is a photomicrograph (magnification: 20 times) showing another portion of the sample of Example 1.

FIG. 14 is a photomicrograph (magnification: 20 times) showing a further portion of the sample of Example 1.

FIG. 15 is a photomicrograph (magnification: 20 times) showing the sample of Example 2.

FIG. 16 is a photomicrograph (magnification: 20 times) showing another portion of the sample of Example 2.

FIG. 17 is a photomicrograph (magnification: 20 times) showing a further portion of the sample of Example 2.

FIG. 18 is a photomicrograph (magnification: 10 times) showing the sample of Example 3.

FIG. 19 is a photomicrograph (magnification: 20 times) showing a portion of the sample of Example 3.

### DESCRIPTION OF THE PREFERRED EMBODIMENTS

[1] Hard Tissue

Hard tissues for samples are not particularly restricted, but may be, for instance, a living hard tissue, a first composite composed of a scaffold constituted by a living hard tissue replacement and cells, a second composite composed of a scaffold constituted by a living hard tissue replacement and a living hard tissue, a third composite composed of a scaffold constituted by a living hard tissue replacement, cells and a living hard tissue, etc. The hard tissue may be any one of them.

(1) Living Hard Tissue

The living hard tissues may be bones, teeth, joints, calculus, etc. The living hard tissue may contain soft tissues such as fibrous tissues, cartilages, etc.

(2) First Composite

The first composite comprises a scaffold (which is constituted by a living hard and/or soft tissue replacement) and cells, and can be used not only as a material for filling and/or mending defects, but also as a material for differentiating and proliferating cells. The living hard tissue replacement may generally be hard materials used for artificial bones, artificial tooth roots, bone fillers, etc., typically calcium compounds. The calcium compounds may be apatites such as hydroxyapatite, fluoroapatite and apatite carbonate, dicalcuim phosphate, tricalcium phosphate, tetracalcium phosphate, octacalcium phosphate, etc.

The living hard tissue replacement may contain soft materials. The soft materials may be known high-molecular materials such as proteins (collagen, albumin, fibrin, etc.); synthesized polymers (polylactic acid, polyglycolic acid, poly-ε-caprolactone, etc.); polysaccharides (starch, alginic acid, etc.), etc. Examples of the living hard tissue replacement composed of a composite of hard materials and soft materials may be composites of calcium compounds and collagen. In the composites, collagen may be cross-linked.

The cells attached to the scaffold constituted by the living hard tissue replacement are not restrictive as long as they can be cultured on the scaffold. The cells may be, for instance, motor cells, liver cells, fibroblasts, urinary cells (for instance, kidney cells, bladder cells, etc.), respiratory cells (for instance, lung cells, alveolus cells, etc.), nerve cells, digestive cells (for instance, stomach cells, small intestine cells, large intestine cells, etc.), circulatory cells (for instance, heart cells, blood vessel cells, etc.), reproductive cells, etc. Stem cells, precursor cells and tumor cells of these cells may also be used.

Among these cells, the motor cells (or their stem cells, precursor cells and tumor cells) are preferable. The motor cells may be, for instance, osteoblasts, osteoblast-like cells, bone cells, cartilage cells, muscle cells, etc. The cells attached to the scaffold may be cultured for a desired period of time. This makes it possible to evaluate how fast cells proliferate in the living hard tissue replacement introduced into a living body.

(3) Second Composite

The second composite comprises a scaffold constituted by the above hard and/or soft living hard tissue replacement and the above living hard tissue.

(4) Third Composite

The third composite comprises a scaffold constituted by the above hard and/or soft living hard tissue replacement, the above cells and the above living hard tissue. The cells attached to the scaffold may be cultured for a desired period of time.

[2] Production of Sample

The method of the present invention for producing samples will be explained in detail below referring to the attached drawings, without intension of restricting the present invention to the depicted methods.

(1) Fixing Step

To stabilize the hard tissue before decalcification, it is preferable to fix the outer shape, internal structure, etc. of the tissue as it is. The fixing of the outer shape, etc. of the tissue is conducted by immersing the hard tissue in a fixative before decalcification.

The fixative may be formaldehyde, paraformaldehyde, glutaraldehyde, etc. These fixatives may be mixed with osmium oxide, acetic acid, phosphoric acid, saturated picric acid, alcohol, etc. Such mixture may be, for instance, a Karnovsky's fixative comprising glutaraldehyde and osmium tetraoxide, a Bouin's fixative comprising formaldehyde, saturated picric acid and glacial acetic acid, a paraformaldehyde/phosphoric acid buffer solution (neutral), etc.

A hard tissue piece cut out to a predetermined shape is immersed in a fixative. The immersion time and temperature are properly selected depending on the types of the hard tissue piece, the fixative, etc. In order that the fixative penetrating into a specimen does not have a concentration gradient, the fixative is preferably stirred. After the outer shape, internal structure, etc. of the hard tissue are fixed, the specimen is washed to remove the fixative. The specimen is preferably washed with flowing water.

(2) Dehydration Step

The fixed specimen is dehydrated. The dehydration improves the permeability of a liquid-penetration-permitting resin into the hard tissue piece. The dehydration is carried out by immersing a specimen in a hydrophilic organic solvent such as ethanol and acetone while stirring. The immersion time and temperature may properly be selected depending on the types and size of the hard tissue piece. During the dehydrating step, the organic solvent is preferably changed several times. In this case, it is preferable to use pluralities of aqueous solutions containing different concentrations of an organic solvent, by successively changing from a lowest-concentration solution to a highest-concentration solution (for instance, 100-% organic solvent).

(3) Primary Immersion Step

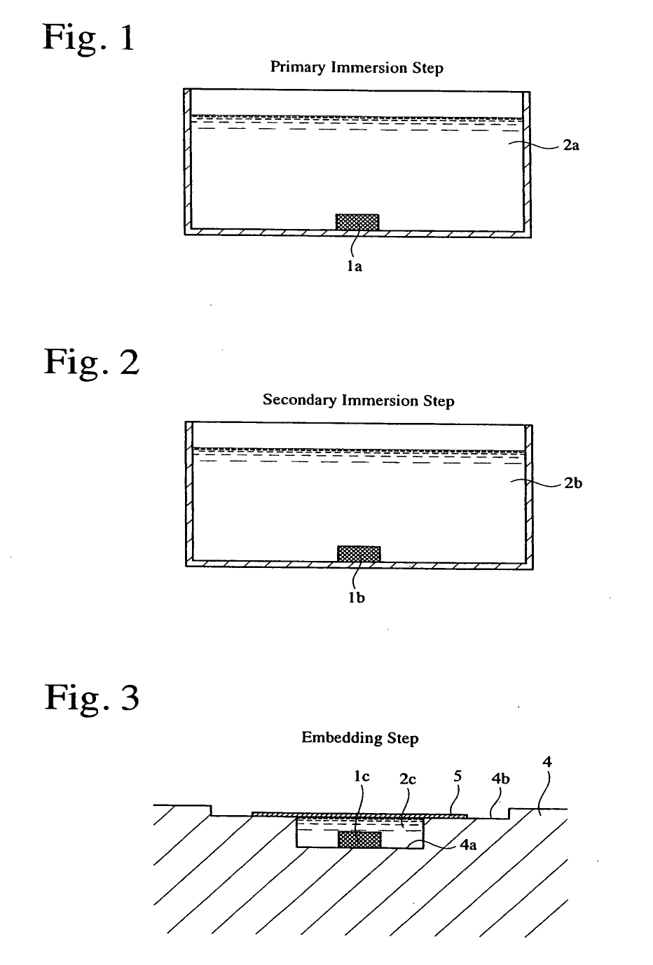

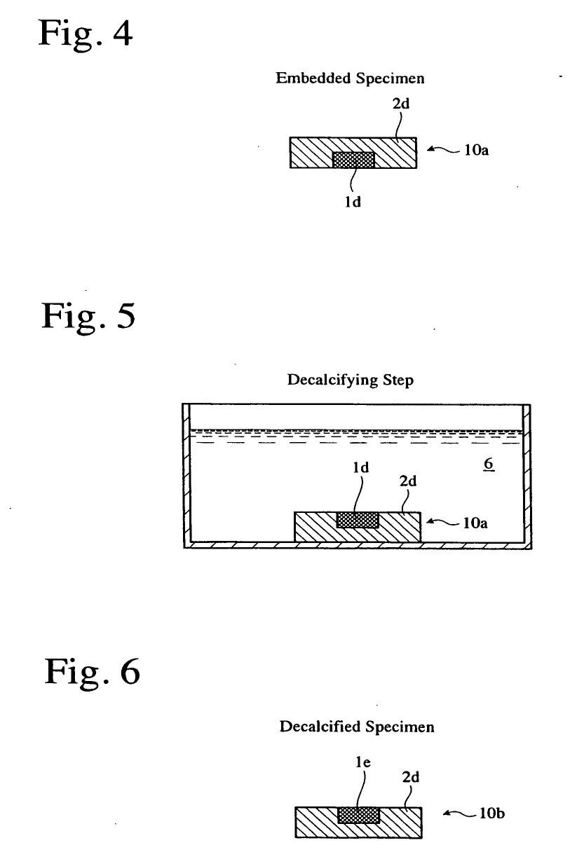

To embed the dehydrated hard tissue piece 1a in a liquid-penetration-permitting resin, it is preferable that the hard tissue piece 1a is first immersed in a mixed liquid (primary immersion liquid) 2a comprising a monomer of a liquid-penetration-permitting resin and an organic solvent as shown in FIG. 1, such that the primary immersion liquid 2a penetrates into the hard tissue piece 1a. The term “liquid-penetration-permitting resin” means a resin, which permits liquids (a decalcifying liquid, a dehydrating liquid, an embedding monomer liquid, etc.) to penetrate into a hard tissue piece 1b after the resin embeds the hard tissue 1b as shown in FIG. 4, for instance.

Monomers used in the primary immersion step may be the same as or different from those used in a secondary immersion step and an embedding step as long as they can form liquid-penetration-permitting resins, though the same monomers are preferable. The details of the monomer of the liquid-penetration-permitting resin will be described below. The organic solvent may be ethanol, acetone, etc. Though not particularly restricted, the concentration of the monomer in the primary immersion liquid 2a is preferably 30-70% by mass. The primary immersion time may be properly selected based on the size of the hard tissue piece 1a, etc. The primary immersion temperature may be room temperature. The primary immersion may be conducted in vacuum.

(4) Secondary Immersion Step

After the primary immersion, the hard tissue piece 1b is preferably immersed in a secondary immersion liquid 2b comprising a monomer and a polymerization initiator without an organic solvent. The secondary immersion time may generally be within 24 hours, and preferably 16-22 hours. The secondary immersion temperature may be room temperature. Incidentally, the monomer used in the secondary immersion may be the same as the monomer in the primary immersion.

The monomer of the liquid-penetration-permitting resin is preferably a monomer curable within a short period of time and capable of forming a high-light-transmittance, liquid-penetration-permitting resin that is not deteriorated for a long period of time. Such monomers may be (meth)acrylic acids and their derivatives, particularly (meth)acrylates or hydroxyalkyl(meth)acrylates, particularly hydroxyalkyl(meth)acrylates. The preferred hydroxyalkyl(meth)acrylates include 2-hydroxymethyl(meth)acrylate, 2-hydroxyethyl(meth)acrylate, 2-hydroxypropyl(meth)acrylate, 2-hydroxybutyl(meth)acrylate, etc. Among them, 2-hydroxymethyl(meth)acrylate and 2-hydroxyethyl(meth)acrylate are preferable, and 2-hydroxyethyl methacrylate (HEMA) is particularly preferable. The (meth)acrylates may be methyl(meth)acrylate, ethyl(meth)acrylate, propyl(meth)acrylate, etc. These monomers may be used alone or in combination. The monomer may contain a plasticizer, if necessary. The plasticizer may be, for instance, a hydroxyether. Commercially available monomers of the liquid-penetration-permitting resins include Technovit 7100 and 8100 available from Heraeus Kulzer of Germany, Historesin Plus available from Leica Microsystems of Germany, JB-4 available from Polyscience of the U.S., etc.

The polymerization initiator is preferably benzoyl peroxide (BPO, LUCIDOL®), etc. Commercially available monomers such as Technovit are accompanied by best-matched polymerization initiators. The amount of the polymerization initiator used may be properly changed depending on combinations with the monomers, and in a case where HEMA is combined with BPO, HEMA/BPO is preferably 100/0.5-100/1.5, for instance, 100/1 by mass. The secondary immersion liquid may contain an auxiliary catalyst, which may be, for instance, those capable of generating chloride ions. Commercially available monomers such as Technovit are accompanied by best-matched auxiliary catalysts.

(5) Embedding Step

As shown in FIG. 3, with the secondarily immersed hard tissue piece 1c placed in a cavity 4a of a mold 4 (for instance, Histoform available from Heraeus Kulzer), an embedding liquid 2c comprising the secondary immersion liquid (the monomer of the liquid-penetration-permitting resin+the polymerization initiator) and a curing accelerator is charged into the cavity 4a, which is then sealed by a cover plate 5. In this case, to prevent the air, which may hinder the solidification of the embedding liquid 2c, from entering, the cavity 4a is filled with the embedding liquid 2c up to a height in alignment with the upper surface 4b of the mold cavity 4a, such that there is no gap between the embedding liquid 2c and the cover plate 5. The curing accelerator may be a solution of barbituric acid derivatives, aromatic amines, N,N-dimethylaniline in styrene, etc. Commercially available monomers such as Technovit are accompanied by best-matched curing accelerators. With the curing accelerator, the monomer can be polymerized at a relatively low temperature. Particularly in a system using BPO as the polymerization initiator, a barbituric acid derivative as the curing accelerator, and an auxiliary catalyst capable of generating chloride ions, the monomer can be fully polymerized by leaving it at room temperature for about 1 hour, and then by warming at 35-45° C. for about 1 hour. Accordingly, when the monomer is warmed in the state shown in FIG. 3, it is polymerized to provide a specimen (embedded specimen) 10a having a hard tissue piece 1c embedded in a liquid-penetration-permitting resin 2d as shown in FIG. 4.

(6) Decalcifying Step

To decalcify the hard tissue piece 1d embedded in the liquid-penetration-permitting resin 2d, the specimen 10a comprising the hard tissue piece 1d and the resin 2d is preferably immersed in a decalcifying liquid 6 comprising an organic acid, an inorganic acid and/or a chelating agent as shown in FIG. 5. Specific examples of the decalcifying liquid 6 include an aqueous solution of formic acid having a concentration of about 5-10% by mass, an aqueous solution of nitric acid having a concentration of about 5-8% by mass, an aqueous solution of ethylenediaminetetraacetic acid (EDTA) having a concentration of about 10% by mass, etc. The decalcifying liquid 6 containing an organic acid may contain formalin or an alcohol. The decalcifying liquid 6 containing formic acid may further contain hydrochloric acid or citric acid.

The specimen 10a having the hard tissue piece 1d embedded in the liquid-penetration-permitting resin 2d is preferably immersed in the decalcifying liquid 6 for 3-30 days, though changeable depending on the size of the hard tissue piece 1d, etc. The decalcifying liquid 6 penetrates into the embedded hard tissue piece 1d to dissolve away calcium components. The decalcifying liquid 6 is used preferably in an amount of 10 times or more the embedded hard tissue piece 1d by volume. If necessary, the decalcifying liquid 6 may be stirred or shaken, or voltage may be applied to the decalcifying liquid 6, to shorten the decalcifying time. A specimen 10b having the resultant the decalcified hard tissue piece 1e (shown in FIG. 6) is preferably washed with water, alcohol, a water-alcohol mixed liquid, etc.

(7) Re-Embedding Step with Resin

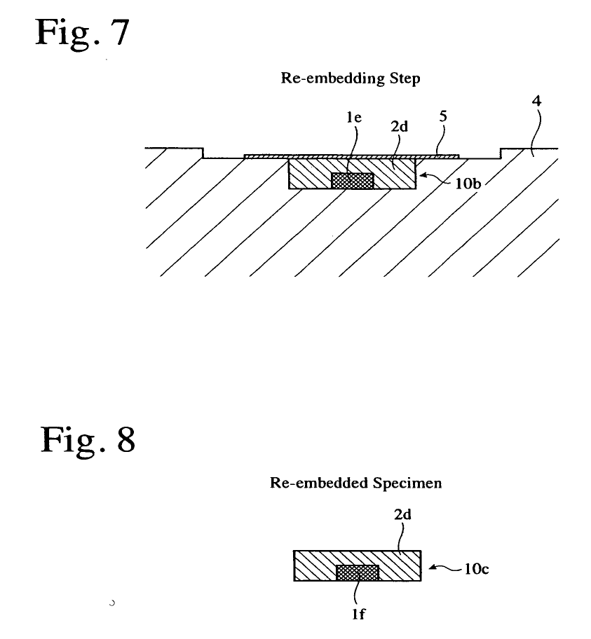

The decalcified hard tissue piece 1e (shown in FIG. 6) has pores left after calcium components are dissolved away. Accordingly, the pores of the decalcified hard tissue piece 1e are preferably filled with a resin 2d again, though not indispensable. As long as they can enter into the pores of the decalcified hard tissue piece 1e, re-embedding resin monomers are not particularly restricted, but they may be preferably the same as the above monomers of the liquid-penetration-permitting resins. The re-embedding treatment with the liquid-penetration-permitting resin is preferably carried out, like above, by washing a specimen 10b comprising the decalcified hard tissue piece 1e, subjecting it to dehydration, primary immersion and secondary immersion, placing it in a cavity 4a of a mold 4 as shown in FIG. 7, charging an embedding liquid into the cavity 4a, warming it under the same conditions as in the step (5) to polymerize the monomer.

(8) Fixing-to-Supporting Block Step

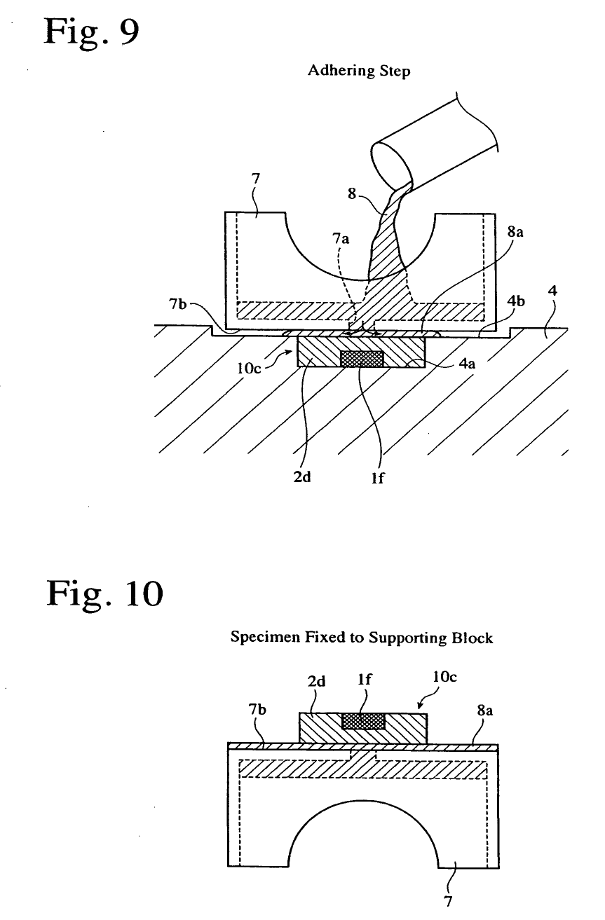

A specimen 10c comprising the re-embedded, decalcified hard tissue piece 1f (shown in FIG. 8) is preferably fixed to a supporting block, so that it is made sliceable by a microtome, etc. For this purpose, as shown in FIG. 9, for instance, it is preferable to charge the re-embedded specimen 10c into the cavity 4a of the same mold 4 as above, hold a hollow supporting block 7 having a hole 7a communicating with a support surface 7b (for instance, “Histoblock” available from Heraeus Kulzer) above the upper surface 4b of the mold cavity 4a, in which the re-embedded specimen 10c is placed, and introduce an adhesive 8 (for instance, “Technovit 3040” available from Heraeus Kulzer mainly comprising methylmethacrylate) into the supporting block 7. The adhesive 8 fills a gap between the upper surface 4b of the mold cavity 4a and the support surface 7b of the supporting block 7, thereby forming an adhesive layer 8a bonding the re-embedded specimen 10c to the support surface 7b. Thus, as shown in FIG. 10, the re-embedded specimen 10c is firmly fixed to the supporting block 7.

(9) Production of Thin Section

The re-embedded, decalcified hard tissue piece 1f of the specimen 10c fixed to the supporting block 7 is sliced by a microtome, etc. to obtain a sample (thin section) for microscopic observation. The thin section is preferably as thick as 0.5-10 μm. The resultant thin section is thus floated on the water for extension treatment, placed on a slide glass, etc., and dried. After stained, it is sealed in a cover glass, etc. to obtain a decalcified tissue sample.

The present invention will be explained in further detail by Examples below, without intension of restricting the present invention thereto.

### EXAMPLE 1

Primary osteoblasts obtained from a skull bone of a newborn rat were attached to hydroxyapatite having a diameter of 5 mm, a thickness of 2 mm, and a porosity of 50% before attaching the cells, and cultured. The cell-attached hydroxyapatite was immersed in a 4-%-by-mass formaldehyde/phosphoric acid buffer solution kept at room temperature for 1 week, to fix the cell tissue to the hydroxyapatite. The fixed specimen was washed with flowing water, immersed in aqueous ethanol solutions of 70% and 96%, respectively, by volume at room temperature for 2 hours each, and then immersed in anhydrous ethanol at room temperature for 1 hour for dehydration.

The dehydrated specimen was immersed in a primary immersion liquid 2a comprising a main ingredient of Technovit 7100 mainly composed of HEMA and including an auxiliary catalyst capable of generating chloride ions, and anhydrous ethanol at an equal volume ratio, at room temperature for 2 hours. The primarily immersed hard tissue piece 1b was then immersed in a secondary immersion liquid 2b at room temperature for 20 hours, and anhydrous ethanol was removed. The secondary immersion liquid 2b comprised the main ingredient of Technovit 7100 and a polymerization initiator, which was a hardener I (BPO with a 20-%-by-mass water content) of Technovit 7100, at a ratio (main ingredient/hardener I) of 100/1 by mass.

The hard tissue piece 1c impregnated with the secondary immersion liquid 2b was charged into a cavity 4a of an embedding mold 4 (Histoform available from Heraeus Kulzer) as shown in FIG. 3, and a embedding liquid 2c comprising the main ingredient of Technovit 7100, the above polymerization initiator, and a curing accelerator, which was a hardener II of Technovit 7100 containing barbituric acid derivatives, at a ratio (secondary immersion liquid /hardener II) of 15/1 by volume was introduced into the cavity 4a, so that the hard tissue piece 1c was immersed in the embedding liquid 2c. After leaving it at room temperature for 1 hour, the hard tissue piece 1c in the embedding liquid 2c was warmed at 37° C. for 1 hour, to polymerize the main ingredient of Technovit 7100.

A specimen 10a having the hard tissue piece 1d embedded in a resin 2d formed from Technovit 7100 was taken out of the mold 4, and immersed in a 5-%-by-mass aqueous formic acid solution at room temperature for 5 days as shown in FIG. 5, to decalcify the hard tissue piece 1d. A specimen 10b having a decalcified hard tissue piece 1e was washed with flowing water, successively dehydrated with a 70-%-by-volume aqueous ethanol solution, a 96-%-by-volume aqueous ethanol solution and anhydrous ethanol, and re-embedded in the resin of Technovit 7100 using the above primary immersion liquid, secondary immersion liquid and embedding liquid in the same manner as above.

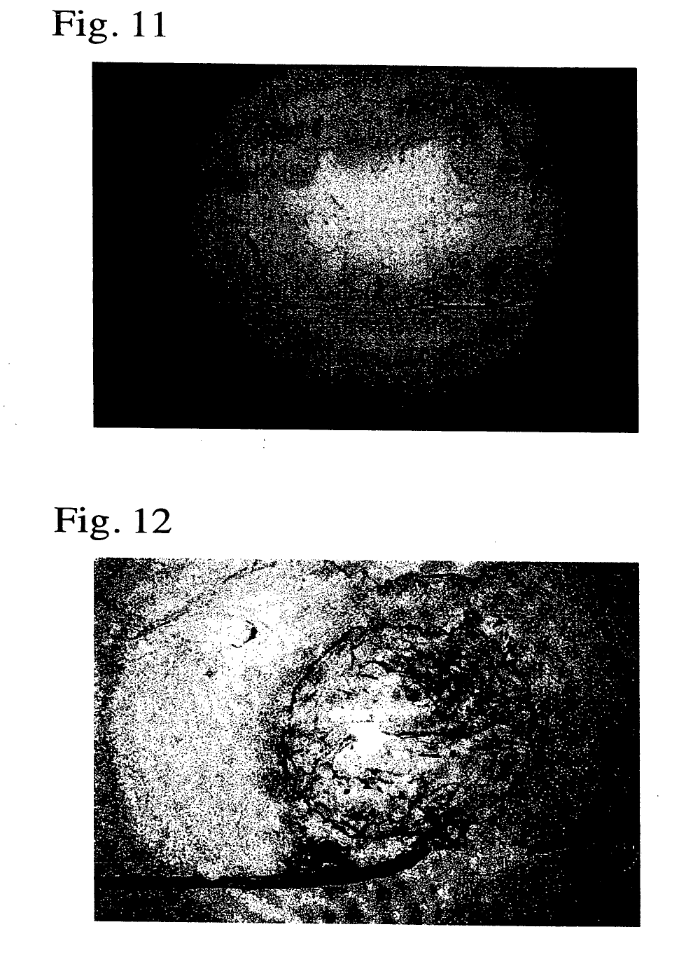

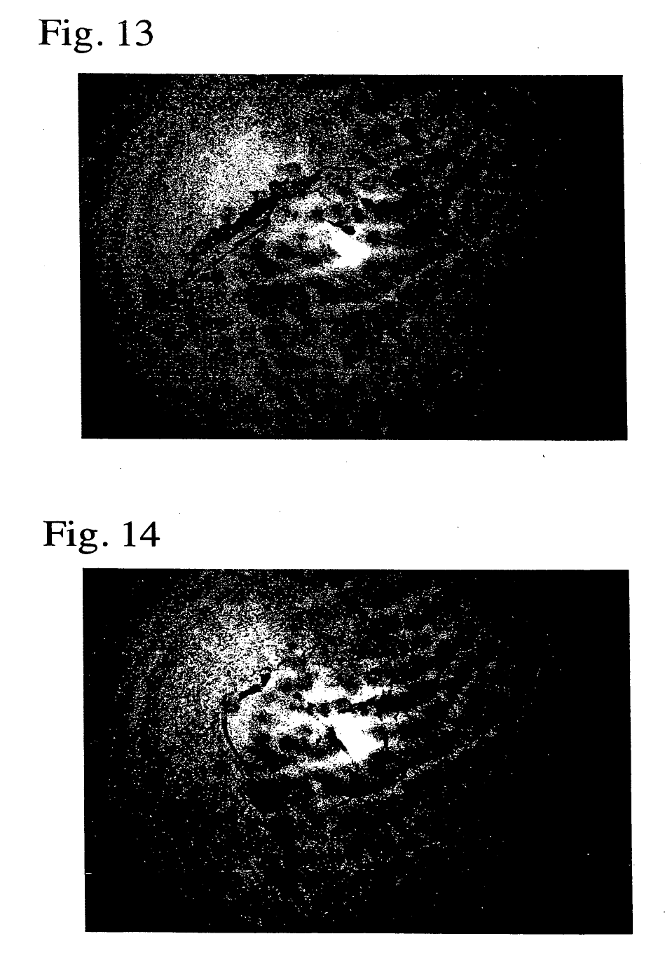

The re-embedded specimen 10c was fixed to a supporting block 7 (Histoblock available from Heraeus Kulzer) with an adhesive 8 (Technovit 3040 available from Heraeus Kulzer) as shown in FIG. 9. The re-embedded specimen 10c fixed to the supporting block 7 (see FIG. 10) was sliced to a thickness of 4 μm by a microtome, and the resultant thin section was expanded on distilled water, placed on a slide glass, and dried at 60° C. for 15 minutes. It was then stained with hematoxilin-eosin (HE), and sealed by a cover glass. The resultant sliced sample of the cell-containing, decalcified hydroxyapatite was observed by an optical microscope. FIGS. 11-14 are photomicrographs of the sample tissue. FIG. 11 shows the entire tissue of the sample at a magnification of 4 times, and FIGS. 12-14 are enlarged photographs (magnification: 20 times) of each portion of the tissue shown in FIG. 11. It is clear from FIGS. 11-14 that a fine tissue structure is clearly observed in the sample produced by the method of the present invention, suggesting that the entire fine structure of the hard tissue was kept unbroken.

### EXAMPLE 2

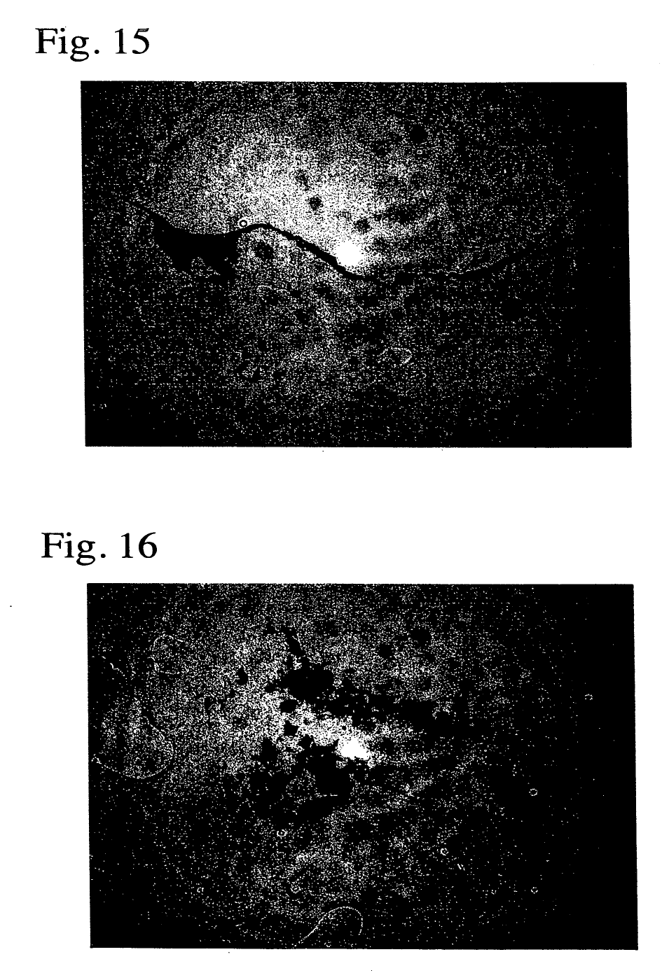

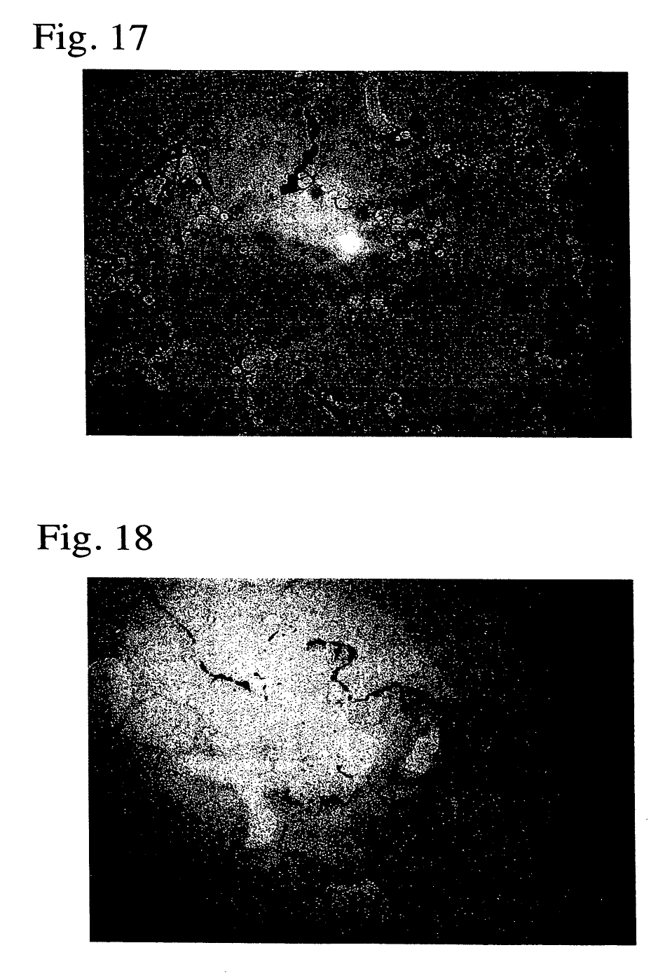

A sliced sample of cell-containing, decalcified hydroxyapatite was produced in the same manner as in Example 1, except for using ultra-porous hydroxyapatite (HAp-S) having a diameter of 5 mm, a thickness of 2 mm, and a porosity of 85% before attaching the cells, to which commercially available clonal osteoblasts [HOS (human osteosarcoma) cells] were attached and cultured. FIGS. 15-17 are optical photomicrographs of the resultant sample.

### EXAMPLE 3

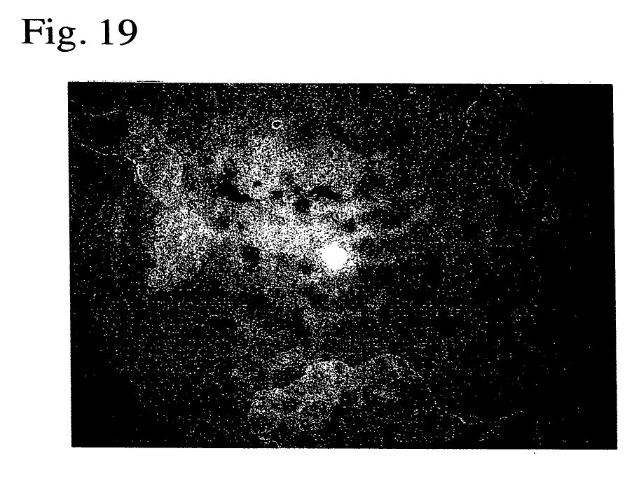

A sliced sample of cell-containing, decalcified hydroxyapatite was produced in the same manner as in Example 2 except for dying it with toluidine blue. FIGS. 18 and 19 are optical photomicrographs of the resultant sample.

As shown in FIGS. 15-19, even when the ultra-porous hydroxyapatite was used, a fine tissue structure was clearly observed as in the case of using hydroxyapatite having usual porosity (Example 1).

### EFFECT OF THE INVENTION

Because the present invention decalcifies a hard tissue after embedding in a liquid-penetration-permitting resin, it can produce a decalcified hard tissue sample simply at a low cost while keeping the fine structure of the hard tissue. The method of the present invention with such feature is particularly suitable for the production of decalcified hard tissue samples of artificial bone, etc., to which cells, etc. are attached and cultured.

The present disclosure relates to subject matter contained in Japanese Patent Application No. 2004-281642 filed on Sep. 28, 2004, which is expressly incorporated herein by reference in its entirety.

What is claimed is:

1. A method for producing a decalcified hard tissue sample comprising embedding said hard tissue in a liquid-penetration-permitting resin, and then decalcifying it.

2. The method for producing a decalcified hard tissue sample according to claim 1, wherein said decalcified, embedded hard tissue is re-embedded in a resin.

3. The method for producing a decalcified hard tissue sample according to claim 1, wherein said hard tissue is a living hard tissue, a first composite comprising a scaffold constituted by a living hard tissue replacement and cells, a second composite comprising said scaffold and said living hard tissue, or a third composite comprising said scaffold, said cells and said living hard tissue.

4. The method for producing a decalcified hard tissue sample according to claim 3, wherein said living hard tissue replacement is made of a calcium compound.

5. The method for producing a decalcified hard tissue sample according to claim 4, wherein said calcium compound is hydroxyapatite.

6. The method for producing a decalcified hard tissue sample according to claim 3, wherein said cells are motor cells.

7. The method for producing a decalcified hard tissue sample according to claim 6, wherein said motor cells are at least one selected from the group consisting of osteoblasts, osteoblast-like cells, bone cells, cartilage cells, muscle cells, and their stem cells, precursor cells and tumor cells.

8. The method for producing a decalcified hard tissue sample according to claim 1, wherein said liquid-penetration-permitting resin is a polymer of hydroxyalkyl(meth)acrylate.

9. The method for producing a decalcified hard tissue sample according to claim 8, wherein said hydroxyalkyl(meth)acrylate is 2-hydroxyethyl methacrylate.
